(19) United States
(12) Patent Application Publication
(10) Pub. No.: US 2004/0143401 A1
(43) Pub. Date: Jul. 22, 2004

(54) Methods and apparatus for discovering, identifying and comparing biological activity mechanisms

(76) Inventor: John W. Elling, Santa Fe, NM (US)

Correspondence Address: FINNEGAN, HENDERSON, FARABOW, GARRETT & DUNNER, LLP, 1300 I STREET, NW, WASHINGTON, DC, 20005, US

(21) Appl. No.: 10/473,626

(22) PCT Filed: Apr. 2, 2002
(86) PCT No.: PCT/US02/10546

(51) Int. Cl. C12Q 1/68; G06F 19/00; G01N 33/48; G01N 33/50

(52) U.S. Cl. 702/19; 435/6

(57) ABSTRACT

Provided herein are methods and devices for the assessment and identification of cellular biological activity mechanisms; the assessment and identification of the changes in cellular biological activity mechanisms caused by cellular perturbations; the assessment and identification of the cellular function, or biological activity mechanisms of genes and gene products and; and the identification of the many genes and their products that collectively act together in a biological mechanism.

### RELATED APPLICATIONS

Benefit of priority is claimed to U.S. Provisional Application Serial No. 60/281,197, filed Apr. 2, 2001, to John W. Elling, entitled “METHOD AND APPARATUS FOR DISCOVERING, IDENTIFYING AND COMPARING BIOLOGICAL ACTIVITY MECHANISMS.” Where permitted the content of the provisional application is incorporated herein by reference in its entirety.

### FIELD OF THE INVENTION

This invention relates to methods and devices for: (1) the assessment and identification of cellular biological activity mechanisms; (2) the assessment and identification of the changes in cellular biological activity mechanisms caused by cellular perturbations; (3) the assessment and identification of the cellular function, or biological activity mechanisms of genes and gene products and; and (4) the identification of the many genes and their products that collectively act together in a biological mechanism.

### BACKGROUND OF THE INVENTION

The two primary costs and bottlenecks in preclinical drug discovery are discovering high quality, “druggable” targets and finding active compounds against those targets that can be used as drugs. Druggable targets are components, typically proteins, of a cellular pathway, that are involved in a disease state and, whose function can be modified with compounds (small organic molecules) that can be used as a drug.

All existing drugs on the market act on only about 400 distinct targets in the body. See MIT Technology Review, September/October 2000, “The Great Gene Grab,” pp. 50-54. These targets are the critical enzymes and other proteins that can be addressed in treating various diseases. For example, the drug Allopurinol is used to treat gout by inhibiting the enzyme Xanthine oxidase, which is involved in the production of uric acid. Another of many possible examples is the drug Captopril, used to treat hypertension, which inhibits the Angiotensin converting enzyme.

Scientists' best guess is that there may be only 5000 druggable targets overall. Pharmaceutical firms are racing to characterize the human genome and proteome in order to identify druggable targets. To progress in this quest, it is necessary to identify what proteins are encoded by the 35,000 human genes. There may be as many as one million proteins present in the body. Drug discovery must then determine under what conditions (in which cells and when) specific proteins are manufactured and in what biological pathways they participate. Finally, it must be determined which of these proteins are appropriate disease related targets against which to develop new drugs.

With a target in hand, it is highly desirable to rapidly identify and optimize compounds to act as drugs against such a target. Combinatorial chemistry and high throughput screening permits the identification of large numbers of bioactive compounds which might be useful as drugs. Now the bottleneck is selection of an active compound that is also bioavailable and will not cause undesirable side effects. The cost of evaluating and optimizing the pharmacokinetics, pharmacodynamics, and side effects of active compounds is huge. Too many compounds fail in late stages of drug discovery and clinical trial after enormous investment. Accordingly, cost-effective methods are needed for the selection of an active compound that is also bioavailable and will not cause undesirable side effects. The present invention satisfies this need and provides related advantages as well.

### SUMMARY OF THE INVENTION

Provided herein are methods of identifying the biological mechanisms affected by a selected gene, comprising culturing a first reference cell under reproducible conditions; processing the first reference cell through an assay in the presence of a perturbation; collecting one or more images of the first cell to detect a first cell assay response to the respective perturbation; culturing a second cell under the reproducible conditions of step a), wherein the first reference cell and the second test-cell are the same cell species, and the second test-cell is altered to modify the expression of the protein encoded by the selected gene; processing the second test-cell through the assay of step b) in the presence of the same perturbation; collecting one or more images of the second cell to detect a second test-cell assay response to the respective perturbation; comparing the one or more images obtained of the first reference cell to the one or more images obtained of the second altered test-cell to identify assay response image changes between the first reference cell and the second test-cell, wherein the assay response image changes correspond to the biological mechanisms affected by the selected gene. These methods can further comprise repeating steps a) through f) above; with a multiplicity of perturbations; and comparing the multiplicity of images obtained of the first reference cell to the multiplicity of images obtained of the second altered test-cell to identify assay response image changes between the first reference cell and the second test-cell, wherein assay response image changes can be used to link the biological mechanisms affected by the selected gene with the biological mechanisms affected by the perturbations.

Also provided are methods of producing a fingerprint of assay responses caused by a perturbation, comprising culturing a first reference cell under reproducible conditions; processing the first reference cell through a multiplicity of assay experiments in the absence of a perturbation; collecting one or more images of the first reference cell to detect a first reference cell assay response to the respective assays; culturing a second test-cell under the reproducible conditions of step a), wherein the first reference cell and the second test-cell are the same cell species; processing the second test-cell through the same multiplicity of assay experiments of step b) in the presence of a perturbation; collecting one or more images of the second test-cell to detect a second test-cell assay response to the respective perturbation; comparing the one or more images obtained of the first reference cell to the one or more images obtained of the second test-cell to identify assay response image changes between the first reference cell and the second test-cell, wherein the assay response image changes correspond to a fingerprint of assay responses caused by the perturbation. These methods can further comprise repeating steps a) through g); with a multiplicity of perturbations; and yet further comprise identifying shared patterns of assay response image changes between the multiplicity of perturbations and identifying within the shared patterns, a specific sub-pattern of assay response image changes, wherein the sub-pattern of assay response image changes corresponds to an individual biological mechanism or a subset of all biological mechanisms affected by the subgroup of perturbations. For this embodiment, the specific sub-pattern of assay response image changes can be identified using one or more statistical clustering methods, wherein the one or more statistical clustering methods can be selected from the group consisting of fuzzy-clustering and multi-domain clustering.

In each of the above-described methods, the perturbation can be selected from any one or more of the forces selected from the group consisting of chemical, biological, mechanical, thermal, electromagnetic, gravitational, nuclear, and temporal; as well as treatment with a test-compound. The test-compound can be known to modulate one or more known biological mechanisms. Likewise, the multiplicity of perturbations can be treatment of the cells with a multiplicity of test-compounds, wherein the multiplicity of test-compounds are each known to modulate one or more known biological mechanisms.

In the above-described methods, the first reference cell can be labeled with one or more imaging reagents corresponding to the respective assay, and the second test-cell can be labeled with the same one or more imaging reagents of step b); the steps a) through g) can be repeated for a multiplicity of different imaging reagents; the one or more imaging reagents can be selected from any combination of cellular stains and molecular labels; and the images can be digitally converted to features. The methods can further comprise correlating the assay responses caused by the test-compounds to the biological mechanisms.

Also in the above-described methods, the expression of the protein encoded by the selected gene can be suppressed, such as by knocking out the selected gene, and the like; the expression of the protein encoded by the selected gene can be enhanced; a series of images can be collected over time to assess the temporal behavior of the first and second cells; the images can be collected after multiple times, during the same assay experiment; the cells can be fixed prior to collecting the images; the images can be collected at different times on different assay experiments of the same cell species; the images collected can be of different assay experiments of same cell type subject to the same perturbation at different quantities; the perturbation can be a test-compound administered at different concentrations; the images can be collected from different locations within the first and second cells; the images can be collected from different locations within the assay container containing the first and second cells; the first and second cells can be cell lines; and the assay response image changes can be associated with the respective perturbation and stored in a database.

The methods described above, can further comprise repeating steps a) through f); with a multiplicity of cell types; and comparing the multiplicity of images obtained of the multiplicity of first reference cells to the multiplicity of images obtained of the multiplicity of second altered test-cells to identify assay response image changes between the multiplicity of first reference cells and the multiplicity of second test-cells, wherein assay response image changes correspond to the biological mechanisms affected by the selected gene in the particular cell type in which a change is detected. The methods can further comprise repeating steps a) through f); with a multiplicity of cell types; and comparing the images obtained of the multiplicity of first reference cell types to the images obtained of the multiplicity of second altered test-cell types to identify assay response image changes that differ between the second test-cell types, wherein assay response image changes correspond to the biological mechanisms affected by the selected gene in the particular cell type. Also provided are imaging devices suitable for conducting the methods provided herein.

Also provided herein are methods of creating a library of patterns of assay response changes in cell lines resulting from assaying known cellular perturbations (e.g., addition of compounds) and then comparing the pattern from the perturbation or gene being investigated to the library to find similarities. The method of generating the library of patterns includes assaying biologically active perturbations (e.g., chemical test-compounds) on cell lines through one or more assays designed to identify the presence and magnitude of the biological effect of the particular perturbation (e.g., compound) in the assay. Each assay response can range from a single value to a multitude of values and at a single point in time or over the course of time. In a particular embodiment, the assay responses are images of living cells. The responses obtained from each of the assays of each of, for example, known biologically active chemical compounds is used to form a pattern, or fingerprint, of assay responses that describe the biological activities exhibited by such compounds on particular types of cells.

The assays used allow observation of the change in behavior of living cells. When the assay involves observation of the cell behavior through an image of the cell, it is necessary to create an assay in which a cell type is cultivated and then imaged with an imaging reagent that allows the targeted biological functionality inside the cells to be visualized (for example, a stain that marks the location of a particular protein in the cells under investigation). First a culture of living cells is created and dispensed into the assay vessel. Next, typically, the environmental perturbation (test-compounds) under investigation will be introduced to the cell culture under investigation and the experiment waits for the perturbation to change the biological activity of the cells. Next, typically, an imaging reagent is introduced to the cell culture and images of the cells are collected and analyzed. The change in the biological activity of the cells caused by the particular perturbation(s) results in a change in the images of the cells when compared to the images of the same cells that were not treated with any perturbation. The image changes are considered the assay response. The assay response provides information on the affect of each tested perturbation (e.g., test-compound) on one or more of the biological mechanisms that affect the biological functionality of the cells being visualized with the particular imaging reagent and imaging system.

A system to observe a wide range of changes of many different cellular mechanisms in many different types of cells is created by running a large number of assays comprising a wide range of cell lines and intercellular imaging reagents (e.g., stains). Each type of cell, cultivated and tested under a specific set of procedures, and optionally labeled with one or more imaging reagents (e.g., fluorescent stains) of a molecular or structural component of a cell, is defined to be a single assay.

Methods are also provided herein to generate a comprehensive catalogue of every affectable cellular metabolic pathway and create a link between those pathways and their interrelated genes, proteins and diseases; Methods are provided herein to automate cellular assays and their result analysis in order to find and characterize cellular metabolic pathways; Methods are provided herein to provide a map of cellular metabolic pathways; Methods are provided herein to observe the response of living cells to perturbations in their metabolism and use these changes to identify individual cellular biological pathways to provide a map of cellular metabolic pathways; Methods are provided herein to create a large library of cellular changes by assaying a large number of biologically active compounds with known cell lines, and digitizing the responses; Methods are provided herein to statistically analyze, or “mine,” the created library for responses to find signatures of individual pathways; Methods are provided herein to compare responses from compounds being investigated for therapeutic value or genes being investigated for relevance to a disease state to signatures mined from the created library to identify the biological pathways being affected by the compound or gene under investigation; Methods are provided herein to identify the specific cellular metabolic pathways corresponding to each discovered signature.

### BRIEF DESCRIPTION OF THE DRAWINGS

FIG. 1 is a flow chart illustrating an exemplary set of processes performed (either manually or using automated high throughput assays and devices) in a laboratory to carry out a cellular assay that is designed to image the normal internal structure and/or activity of untreated cells.

FIG. 2 is a flow chart illustrating an exemplary set of processes performed (either manually or using automated high throughput assays and devices) in a laboratory to assess the change in images of cells in an assay that results from the effect of a particular compound.

FIG. 3 is an exemplary matrix representation of the library of descriptors of reference image changes, in which each of the assays defines a row in the matrix, each of the tested compounds represents a column in the matrix, and the library of reference image changes is represented by a set of descriptors.

### DETAILED DESCRIPTION OF THE INVENTION

A. Definitions

Unless defined otherwise, all technical and scientific terms used herein have the same meaning as is commonly understood by one of skill in the art to which the invention(s) belong. All patents, patent applications, published applications and publications, websites and other published materials referred to throughout the entire disclosure herein, unless noted otherwise, are incorporated by reference in their entirety. Where reference is made to a URL or other such identifier or address, it understood that such identifiers can change and particular information on the internet can come and go, but equivalent information can be found by searching the internet. Reference thereto evidences the availability and public dissemination of such information.

As used herein the phrase “culturing a cell under reproducible conditions”, or grammatical variations thereof, refers to tightly controlled cellular growth and environmental conditions, to obtain batches that behave identically to each other each time a biological assay is performed on the particular cell type. Such conditions can be achieved using methods and cell culturing devices well-know in the art.

As used herein, the term “perturbation” refers to any environmental change that can alter the biological activity of a cell. Exemplary perturbations include, but are not limited to, any combination of one or more of chemical, biological, mechanical, thermal (e.g., heat shock, and the like), electromagnetic, gravitational, nuclear, or temporal factors, for example. For example, perturbations could include exposure to chemical compounds, including biologically active test-compounds of known biological activity such as therapeutics or drugs, or also compounds of unknown biological activity. Or exposure to biologics that may or may not be used as drugs such as hormones, growth factors, antibodies, or extracellular matrix components. Or exposure to biologics such as infective materials such as viruses that may be naturally occurring viruses or viruses engineered to express exogenous genes at various levels. Bioengineered viruses are one example of perturbations via gene transfer. Other means of gene transfer are well known in the art and include but are not limited to electroporation, calcium phosphate precipitation, and lipid-based transfection.

Physical perturbations could include exposing cells to shear stress under different rates of fluid flow, exposure of cells to different temperatures, exposure of cells to vacuum or positive pressure, or exposure of cells to sonication. Perturbations could also include applying centrifugal force. Perturbations could also include changes in gravitational force, including sub-gravitation (a particular embodiment in outer space). Perturbations could include application of a constant or pulsed electrical current. Perturbations could also include irradiation. Perturbations could also include photobleaching which in some embodiments may include prior addition of a substance that would specifically mark areas to be photobleached by subsequent light exposure. In addition, these types of perturbations may be varied as to time of exposure, or cells could be subjected to multiple perturbations in various combinations and orders of addition. Of course, the type of perturbation used depends upon the application. In a particular embodiment, a multiplicity of perturbations can be achieved by treating cells with a multiplicity of test-compounds.

As used herein, the phrase “altered to modify the expression of the protein encoded by a selected gene” refers to modulation of protein function (e.g., enhancing, inhibiting, knocking-out, and the like), either at the transcription, translation, or post-translation levels, by any means known to those of skill in the art. Test-cells can be altered to modify the expression of the protein encoded by a selected gene using a variety of methods well-known in the art. See, e.g., Brummelkamp et al., Science (online), Mar. 21, 2002, describing a plasmid-based method for knocking out gene function; U.S. Pat. No. 5,772,995 and Capecchi, Nature, 344:105, describing a homologous recombination gene “knock-out” method; U.S. Pat. No. 5,955,330, describing method for enhancing gene expression; U.S. Pat. No. 6,358,932 describing antisense oligonucleotide inhibition of raf gene expression; U.S. Pat. No. 6,331,617, describing positively charged oligonucleotides as regulators of gene expression; U.S. Pat. No. 6,147,279, describing the inhibition of gene expression; U.S. Pat. No. 4,748,119, describing altering, regulating and enhancing gene expression.

A. Creating One Reference Assay (One Type Of Cells; One Stain)

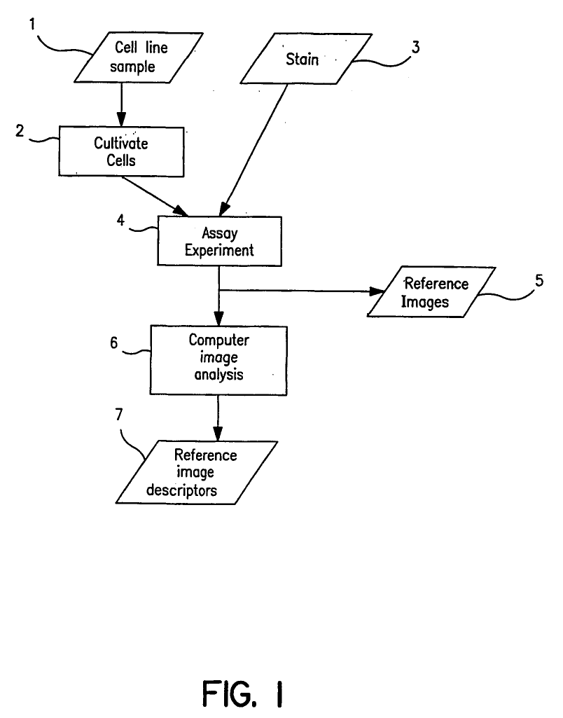

For a particular type of cells, the first step is to culture a batch of such cells under extremely tightly controlled and reproducible conditions. Typically in this process, a sample of the cell line is obtained, as illustrated in FIG. 1 as step 1, and manipulated such that the cells reproduce in a nutrient solution, creating a liquid that contains the cells and nutrients in suspension, as illustrated in FIG. 1 as step 2. As those skilled in the art will appreciate, it is necessary to tightly control the growth conditions, and environmental conditions during growth, to obtain batches that behave identically to each other each time the assay is performed on this cell type. The cultivation of the cells can be automated in order to grow batches of cells under tightly controlled reproducible conditions. Commercial systems for this purpose are well-known and readily available. For example, the Aastrom Replicell (www.aastrom.com) can be used to grow cultures of human cell lines. The Automation Partnership (Cambridge UK) also provides automated equipment for growing culture cells.

In FIG. 1, step 3, an imaging reagent is obtained that is known to be suitable to visualize the desired structures or targets inside the cell line which is being cultured. As used herein, the term “imaging reagent” refers to any agent or molecule that facilitates the imaging of any component of a cell or cell matrix using well-known imaging methods. The term imaging reagent therefore encompasses any stain, label, probe, marker, or the like known to those of skill in the art, so long as it facilitates the imaging of any component of a cell or cell matrix. Conventionally, “stains” are typically used as tags of cell structures and “labels” are typically used for tagging molecules, such as proteins and DNA. For purposes herein, any agent that binds a fluorophore or chromophore, or the like, to a molecular or structural component in or on a cell is useful herein as an imaging reagent. In certain embodiments, chromophores are used to permit imaging in regular light (e.g., white-light imaging). In addition, regular, white-light imaging, infrared imaging and UV imaging of cells are contemplated herein and can be utilized to fingerprint the resulting image without any labeling or staining. In other embodiments, any number of labels can be used in combination with a single cell type in a single experiment, depending on the capabilities of thee imaging instrument. For example, with three filters (usually in a wheel), an imaging instrument can be used to collect three images at three wavelengths and so the resulting composite image of that group of cells has three colors detecting three different cellular components.

For example, the interaction of the imaging reagent (e.g., a fluorescent stain) and the structures or targets in such cells allows one to see an image of these structures or targets. Typically the chosen stain comprises of a component that binds to the desired part of the cell and a component that is optically active by, for example, fluorescing when excited with ultraviolet light. Numerous stains for specific internal components of cells are known in the prior art. For instance, Hoechst dye is frequently used to stain cell nuclei, phalloidin can be used to label filamentous actin and DNasel can be used to label monomeric actin. The fluorescent stain DAPI can be used in cytological analysis involving fluorescence image cytometry as described in embodiments described in U.S. Pat. No. 5,548,661. While the use of DAPI is commonly known in the art it should be appreciated by those skilled in the art that numerous other stains and labeling techniques may be effective for use in cytological and molecular analysis, such as antibodies tagged with fluorescent or chemiluminescent moieties. Other stains such as the densitometric stain, Fuelgen, and Hoesct which may be used with live cells (although more toxic than DAPI) are also described. U.S. Pat. Nos. 4,906,561 and 4,668,618 additionally discusses the use of DAPI and are incorporated by reference. Thioflavin T and thiazole orange are fluorescent stains described in U.S. Pat. No. 4,957,870. Xanthene dyes are disclosed in U.S. Pat. No. 4,933,471 while fluorescently-tagged antibodies are discussed in U.S. Pat. No. 4,983,359 also incorporated by reference. Other fluorescent stains and methods of use thereof are described in U.S. Pat. No. 4,959,301 and 4,987,870 which are also incorporated by reference. Additionally alternate imaging methods which involve the use of DNA-specific, densiometric stains, or other various fluorescent labels and satins such as Feulgen Azure A, chromogen, methyl green, immunohistochemical stains, or ionic stains are described in U.S. Pat. No. 5,548,661, incorporated herein by reference. Several alternative non-fluorescent staining techniques are described in U.S. Pat. Nos. 4,998,284 and 5,016,283.

Other stains are well-known and include the use of a luminophore as described in PCT application WO 98/45704 in which the luminophore may be a florophore such as a polypeptide encoded by and expressed form a nucleotide sequence within the cell or cells. The luminescent polypeptide could also be a green fluorescent protein (GFP) as described in WO98/45704 or GFP mutations described therein.

Likewise, other imaging reagents and systems are well-known and include high-content screens involving the functional localization of the following exemplary macromolecules as described in WO 00/50872. Within this class of high-content screen, the functional localization of macromolecules in response to external stimuli is measured within living cells.

Glycolytic Enzyme Activity Regulation.

In one embodiment of a cellular enzyme activity high-content screen, the activity of key glycolytic regulatory enzymes are measured in treated cells. To measure enzyme activity, indicator cells containing luminescent labeling reagents are treated with test compounds and the activity of the reporters is measured in space and time using cell screening methods provided herein.

In one embodiment, the reporter of intracellular enzyme activity is fructose phosphate, 2-kinase/fructose-2,6-bisphosphatase (PFK-2), a regulatory enzyme whose phosphorylation state indicates intracellular carbohydrate anabolism or catabolism (Deprez et al. (1997) J Biol. Chem. 272:17269-17275; Kealer et al. (1996) FEBS Letters 395:225-227; Lee et al. (1996), Biochemistry 35:6010-6019). The indicator cells contain luminescent reporters comprising a fluorescent protein biosensor of PFK-2 phosphorylation. The fluorescent protein biosensor is constructed by introducing an environmentally sensitive fluorescent dye near to the known phosphorylation site of the enzyme (Deprez et al. (1997), supra; Giuliano et al. (1995), supra). The dye can be of the ketocyanine class (Kessler and Wolfbeis (1991), Spectrochimica Acta 47A: 187-192) or any class that contains a protein reactive moiety and a fluorochrome whose excitation or emission spectrum is sensitive to solution polarity. The fluorescent protein biosensor is introduced into the indicator cells using bulk loading methodology.

Living indicator cells are treated with test compounds, at final concentrations ranging from 10-¹² M to 10-³ M for times ranging from 0.1 s to 10 h. In a particular embodiment, ratio image data are obtained from living treated indicator cells by collecting a spectral pair of fluorescence images at each time point. To extract morphometric data from each time point, a ratio is made between each pair of images by numerically dividing the two spectral images at each time point, pixel by pixel. Each pixel value is then used to calculate the fractional phosphorylation of PFK. At small fractional values of phosphorylation, PFK-2 stimulates carbohydrate catabolism. At high fractional values of phosphorylation, PFK-2 stimulates carbohydrate anabolism.

Protein Kinase A Activity and Localization of Subunits.

In another embodiment of a high-content screen, both the domain localization and activity of protein kinase A (PKA) within indicator cells are measured in response to treatment with test compounds.

The indicator cells contain luminescent reporters including a fluorescent protein biosensor of PKA activation. The fluorescent protein biosensor is constructed by introducing an environmentally sensitive fluorescent dye into the catalytic subunit of PKA near the site known to interact with the regulatory subunit of PKA (Harootunian et al. (1993), Mol. Biol. of the Cell 4:993-1002; Johnson et al. (1996), Cell 85:149-158; Giuliano et al. (1995), supra). The dye can be of the ketocyanine class (Kessler, Wolfbeis (1991), Spectrochimica Acta 47A:187-192) or any class that contains a protein reactive moiety and a fluorochrome whose excitation or emission spectrum is sensitive to solution polarity. The fluorescent protein biosensor of PKA activation is introduced into the indicator cells using bulk loading methodology.

In one embodiment, living indicator cells are treated with test-compounds, at final concentrations ranging from 10-¹² M to 10-³ M for times ranging from 0.1 s to 10 h. In a particular embodiment, ratio image data are obtained from living treated indicator cells. To extract biosensor data from each time point, a ratio is made between each pair of images, and each pixel value is then used to calculate the fractional activation of PKA (e.g., separation of the catalytic and regulatory subunits after cAMP binding). At high fractional values of activity, PFK-2 stimulates biochemical cascades within the living cell.

To measure the translocation of the catalytic subunit of PKA, indicator cells containing luminescent reporters are treated with test compounds and the movement of the reporters is measured in space and time using the cell screening system. The indicator cells contain luminescent reporters comprising domain markers used to measure the localization of the cytoplasmic and nuclear domains. When the indicator cells are treated with a test compounds, the dynamic redistribution of a PKA fluorescent protein biosensor is recorded intracellularly as a series of images over a time scale ranging from 0.1 s to 10 h. Each image is analyzed by a method that quantifies the movement of the PKA between the cytoplasmic and nuclear domains. To do this calculation, the images of the probes used to mark the cytoplasmic and nuclear domains are used to mask the image of the PKA fluorescent protein biosensor. The integrated brightness per unit area under each mask is used to form a translocation quotient by dividing the cytoplasmic integrated brightness/area by the nuclear integrated brightness/area. By comparing the translocation quotient values from control and experimental wells, the percent translocation is calculated for each potential lead compound. The output of the high-content screen relates quantitative data describing the magnitude of the translocation within a large number of individual cells that have been treated with test compound in the concentration range of 10-¹² M to 10-³ M.

High-content screens involving the induction or inhibition of gene expression.

Cytoskeletal Protein Transcription and Message Localization.

Regulation of the general classes of cell physiological responses including cell-substrate adhesion, cell-cell adhesion, signal transduction, cell-cycle events, intermediary and signaling molecule metabolism, cell locomotion, cell-cell communication, and cell death can involve the alteration of gene expression. High-content screens can also be designed to measure this class of physiological response.

In one embodiment, the reporter of intracellular gene expression is an oligonucleotide that can hybridize with the target mRNA and alter its fluorescence signal. In a particular embodiment, the oligonucleotide is a molecular beacon (Tyagi and Kramer (1996) Nat. Biotechnol. 14:303-308), a luminescence-based reagent whose fluorescence signal is dependent on intermolecular and intramolecular interactions. The fluorescent biosensor is constructed by introducing a fluorescence energy transfer pair of fluorescent dyes such that there is one at each end (5′ and 3′) of the reagent. The dyes can be of any class that contains a protein reactive moiety and fluorochromes whose excitation and emission spectra overlap sufficiently to provide fluorescence energy transfer between the dyes in the resting state, including, but not limited to, fluorescein and rhodamine (Molecular Probes, Inc.). In a particular embodiment, a portion of the message coding for β-actin (Kislauskis et al. (1994), J. Cell Biol. 127:441-451; McCann et al. (1997), Proc. Nat. Acad Sci. 94:5679-5684; Sutoh (1982), Biochemistry 21:3654-3661) is inserted into the loop region of a hairpin-shaped oligonucleotide with the ends tethered together due to intramolecular hybridization. At each end of the biosensor a fluorescence donor (fluorescein) and a fluorescence acceptor (rhodamine) are covalently bound. In the tethered state, the fluorescence energy transfer is maximal and therefore indicative of an unhybridized molecule. When hybridized with the mRNA coding for β-actin, the tether is broken and energy transfer is lost. The complete fluorescent biosensor is introduced into the indicator cells using bulk loading methodology.

In one embodiment, living indicator cells are treated with test compounds, at final concentrations ranging from 10-¹² M to 10-³ M for times ranging from 0.1 s to 10 h. In a particular embodiment, ratio image data are obtained from living treated indicator cells. To extract morphometric data from each time point, a ratio is made between each pair of images, and each pixel value is then used to calculate the fractional hybridization of the labeled nucleotide. At small fractional values of hybridization little expression of β-actin is indicated. At high fractional values of hybridization, maximal expression of β-actin is indicated. Furthermore, the distribution of hybridized molecules within the cytoplasm of the indicator cells is also a measure of the physiological response of the indicator cells.

Labeled Insulin Binding to its Cell Surface Receptor in Living Cells.

Cells whose plasma membrane domain has been labeled with a labeling reagent of a particular color are incubated with a solution containing insulin molecules (Lee et al. (1997), Biochemistry 36:2701-2708; Martinez-Zaguilan et al. (1996), Am. J Physiol. 270:CI438-CI446) that are labeled with a luminescent probe of a different color for an appropriate time under the appropriate conditions. After incubation, unbound insulin molecules are washed away, the cells fixed and the distribution and concentration of the insulin on the plasma membrane is measured. To do this, the cell membrane image is used as a mask for the insulin image. The integrated intensity from the masked insulin image is compared to a set of images containing known amounts of labeled insulin. The amount of insulin bound to the cell is determined from the standards and used in conjunction with the total concentration of insulin incubated with the cell to calculate a dissociation constant or insulin to its cell surface receptor.

Whole Cell Labeling of Cellular Compartments

Whole cell labeling is accomplished by labeling cellular components such that dynamics of cell shape and motility of the cell can be measured over time by analyzing fluorescence images of cells.

In one embodiment, small reactive fluorescent molecules are introduced into living cells. These membrane-permeant molecules both diffuse through and react with protein components in the plasma membrane. Dye molecules react with intracellular molecules to both increase the fluorescence signal emitted from each molecule and to entrap the fluorescent dye within living cells. These molecules include reactive chloromethyl derivatives of aminocoumarins, hydroxycoumarins, eosin diacetate, fluorescein diacetate, some Bodipy dye derivatives, and tetramethylrhodamine. The reactivity of these dyes toward macromolecules includes free primary amino groups and free sulfhydryl groups.

In another embodiment, the cell surface is labeled by allowing the cell to interact with fluorescently labeled antibodies or lectins (Sigma Chemical Company, St. Louis, Mo.) that react specifically with molecules on the cell surface. Cell surface protein chimeras expressed by the cell of interest that contain a green fluorescent protein, or mutant thereof, component can also be used to fluorescently label the entire cell surface. Once the entire cell is labeled, images of the entire cell or cell array can become a parameter in high content screens, involving the measurement of cell shape, motility, size, and growth and division.

Plasma Membrane Labeling

In one embodiment, labeling the whole plasma membrane employs some of the same methodology described above for labeling the entire cells. Luminescent molecules that label the entire cell surface act to delineate the plasma membrane.

In a second embodiment subdomains of the plasma membrane, the extracellular surface, the lipid bilayer, and the intracellular surface can be labeled separately and used as components of high content screens. In the first embodiment, the extracellular surface is labeled using a brief treatment with a reactive fluorescent molecule such as the succinimidyl ester or iodoacetamde derivatives of fluorescent dyes such as the fluoresceins, rhodamines, cyanines, and Bodipys.

In a third embodiment, the extracellular surface is labeled using fluorescently labeled macromolecules with a high affinity for cell surface molecules. These include fluorescently labeled lectins such as the fluorescein, rhodamine, and cyanine derivatives of lectins derived from jack bean (Con A), red kidney bean (erythroagglutinin PHA-E), or wheat germ.

In a fourth embodiment, fluorescently labeled antibodies with a high affinity for cell surface components are used to label the extracellular region of the plasma membrane. Extracellular regions of cell surface receptors and ion channels are examples of proteins that can be labeled with antibodies.

In a fifth embodiment, the lipid bilayer of the plasma membrane is labeled with fluorescent molecules. These molecules include fluorescent dyes attached to long chain hydrophobic molecules that interact strongly with the hydrophobic region in the center of the plasma membrane lipid bilayer. Examples of these dyes include the PKH series of dyes (U.S. Pat. Nos. 4,783,401, 4,762701, and 4,859,584; available commercially from Sigma Chemical Company, St. Loius, Mo.), fluorescent phospholipids such as nitrobenzoxadiazole glycerophosphoethanolamine and fluorescein-derivatized dihexadecanoylglycerophosphoetha-nolamine, fluorescent fatty acids such as 5-butyl4,4-difluoro bora-3a,4a-diaza-s-indacene nonanoic acid and 1-pyrenedecanoic acid (Molecular Probes, Inc.), fluorescent sterols including cholesteryl 4,4-difluoro-5,7dimethyl bora-3a,4a-diaza-s-indacene dodecanoate and cholesteryl 1pyrenehexanoate, and fluorescently labeled proteins that interact specifically with lipid bilayer components such as the fluorescein derivative of annexin V (Caltag Antibody Co, Burlingame, Calif.).

In another embodiment, the intracellular component of the plasma membrane is labeled with fluorescent molecules. Examples of these molecules are the intracellular components of the trimeric G-protein receptor, adenylyl cyclase, and ionic transport 81 proteins. These molecules can be labeled as a result of tight binding to a fluorescently labeled specific antibody or by the incorporation of a fluorescent protein chimera that is comprised of a membrane-associated protein and the green fluorescent protein, and mutants thereof.

Endosome Fluorescence Labeling

In one embodiment, ligands that are transported into cells by receptor-mediated endocytosis are used to trace the dynamics of endosomal organelles. Examples of labeled ligands include Bodipy FL-labeled low density lipoprotein complexes, tetramethylrhodarnine transferrin analogs, and fluorescently labeled epidermal growth factor (Molecular Probes, Inc.)

In a second embodiment, fluorescently labeled primary or secondary antibodies (Sigma Chemical Co. St. Louis, Mo.; Molecular Probes, Inc. Eugene, Oreg.; Caltag Antibody Co.) that specifically label endosomal ligands are used to mark the endosornal compartment in cells.

In a third embodiment, endosomes are fluorescently labeled in cells expressing protein chimeras formed by fusing a green fluorescent protein, or mutants thereof, with a receptor whose internalization labels endosomes. Chimeras of the EGF, transferrin, and low density lipoprotein receptors are examples of these molecules.

Lysosome Labeling

In one embodiment, membrane penneant lysosome-specific luminescent reagents are used to label the lysosomal compartment of living and fixed cells. These reagents include the luminescent molecules neutral red, N-(3-((2,4-dinitrophenyl)amino)propyl)-N-(3-aminopropyl)methylamine, and the LysoTracker probes which report intralysosomal pH as well as the dynamic distribution of lysosomes (Molecular Probes, Inc.) In a second embodiment, antibodies against lysosomal antigens (Sigma Chemical Co.; Molecular Probes, Inc.; Caltag Antibody Co.) are used to label lysosomal components that are localized in specific lysosomal domains. Examples of these components are the degradative enzymes involved in cholesterol ester hydrolysis, membrane protein proteases, and nucleases as well as the ATP-driven lysosomal proton pump.

In a third embodiment, protein chimeras comprising a lysosomal protein genetically fused to an intrinsically luminescent protein such as the green fluorescent protein, or mutants thereof, are used to label the lysosomal domain. Examples of these components are the degradative enzymes involved in cholesterol ester hydrolysis, membrane protein proteases, and nucleases as well as the ATP-driven lysosomal proton PUMP.

Cytoplasmic Fluorescence Labeling

In one embodiment, cell permeant fluorescent dyes (Molecular Probes, Inc.) with a reactive group are reacted with living cells. Reactive dyes including monobromobimane, 5-chloromethylfluorescein diacetate, carboxy fluorescein diacetate succinimidyl ester, and chloromethyl tetramethylrhodamine are examples of cell permeant fluorescent dyes that are used for long term labeling of the cytoplasm of cells.

In a second embodiment, polar tracer molecules such as Lucifer yellow and cascade blue-based fluorescent dyes (Molecular Probes, Inc.) are introduced into cells using bulk loading methods and are also used for cytoplasmic labeling.

In a third embodiment, antibodies against cytoplasmic components (Sigma Chemical Co.; Molecular Probes, Inc.; Caltag Antibody Co.) are used to fluorescently label the cytoplasm. Examples of cytoplasmic antigens are many of the enzymes involved in intermediary metabolism. Enolase, phosphofructokinasei and acetyl-CoA dehydrogenase are examples of uniformly distributed cytoplasmic antigens.

In a fourth embodiment, protein chimeras comprising a cytoplasmic protein genetically fused to an intrinsically luminescent protein such as the green fluorescent protein, or mutants thereof, are used to label the cytoplasm. Fluorescent chimeras of uniformly distributed proteins are used to label the entire cytoplasmic domain. Examples of these proteins are many of the proteins involved in intermediary metabolism and include enolase, lactate dehydrogenase, and hexokinase.

In a fifth embodiment, antibodies against cytoplasmic antigens (Sigma Chemical Co.; Molecular Probes, Inc.; Caltag Antibody Co.) are used to label cytoplasmic components that are localized in specific cytoplasmic sub-domains. Examples of these components are the cytoskeletal proteins actin, tubulin, and cytokeratin. A population of these proteins within cells is assembled into discrete structures, which in this case, are fibrous. Fluorescence labeling of these proteins with antibody-based reagents therefore labels a specific subdomain of the cytoplasm.

In a sixth embodiment, non-antibody-based fluorescently labeled molecules that interact strongly with cytoplasmic proteins are used to label specific cytoplasmic components. One example is a fluorescent analog of the enzyme DNAse I (Molecular Probes, Inc.) Fluorescent analogs of this enzyme bind tightly and specifically to cytoplasmic actin, thus labeling a sub-domain of the cytoplasm. In another example, fluorescent analogs of the mushroom toxin phalloidin or the drug paclitaxel (Molecular Probes, Inc.) are used to label components of the actin- and microtubule-cytoskeletons, respectively.

In a seventh embodiment, protein chimeras comprising a cytoplasmic protein genetically fused to an intrinsically luminescent protein such as the green fluorescent protein, or mutants thereof, are used to label specific domains of the cytoplasm. Fluorescent chimeras of highly localized proteins are used to label cytoplasmic subdomains. Examples of these proteins are many of the proteins involved in regulating the cytoskeleton. They include the structural proteins actin, tubulin, and cytokeratin as well as the regulatory proteins microtubule associated protein 4 and cc-actinin.

Nuclear Labeling

In one embodiment, membrane permeant nucleic-acid-specific luminescent reagents (Molecular Probes, Inc.) are used to label the nucleus of living and fixed cells. These reagents include eyanine-based dyes (e.g., TOTO®, YOYO®, and BOBO™), phenanthidines and acridines (e.g., ethidiurn bromide, propidium iodide, and acridine orange), indoles and imidazoles (e.g., Hoechst 33258, Hoechst 33342, and 4′,6-diamidino phenyiindole), and other similar reagents (e.g., 7-aminoactinomycin D, hydroxystilbarnidine, and the psoralens).

In a second embodiment, antibodies against nuclear antigens (Sigma Chemical Co.; Molecular Probes, Inc.; Caltag Antibody Co.) are used to label nuclear components that are localized in specific nuclear domains. Examples of these components are the macromolecules involved in maintaining DNA structure and function. DNA, RNA, histones, DNA polymerase, RNA polymerase, lamins, and nuclear variants of cytoplasmic proteins such as actin are examples of nuclear antigens.

In a third embodiment, protein chimeras comprising a nuclear protein genetically fused to an intrinsically luminescent protein such as the green fluorescent protein, or mutants thereof, are used to label the nuclear domain. Examples of these proteins are many of the proteins involved in maintaining DNA structure and function. Histones, DNA polymerase, RNA polymerase, lamins, and nuclear variants of cytoplasmic proteins such as actin are examples of nuclear proteins.

Mitochondrial Labeling

In one embodiment, membrane permeant mitochondrial-specific luminescent reagents (Molecular Probes, Inc.) are used to label the mitochondria of living and fixed cells. These reagents include rhodamine 123, tetramethyl rosamine, X-1, and the MitoTracker reactive dyes.

In a second embodiment, antibodies against mitochondrial antigens (Sigma Chemical Co.; Molecular Probes, Inc.; Caltag Antibody Co.) are used to label mitochondrial components that are localized in specific mitochondrial domains. Examples of these components are the macromolecules involved in maintaining mitochondrial DNA structure and function. DNA, RNA, histones, DNA polymerase, RNA polymerase, and mitochondrial variants of cytoplasmic macromolecules such as mitochondrial tRNA and rRNA are examples mitochondrial antigens. Other examples of mitochondrial antigens are the components of the oxidative phosphorylation system found in the mitochondria (e.g., cytochrome c, cytochrome c oxidase, and succinate dehydrogenase).

In a third embodiment, protein chimeras comprising a mitochondrial protein genetically fused to an intrinsically luminescent protein such as the green fluorescent protein, or mutants thereof, are used to label the mitochondrial domain. Examples of these components are the macromolecules involved in maintaining mitochondrial DNA structure and function. Examples include histones, DNA polymerase, RNA polymerase, and the components of the oxidative phosphorylation system found in the mitochondria (e.g., cytochrome c, cytochrome c oxidase, and succinate dehydrogenase).

Endoplasmic Reticulum Labeling

In one embodiment, membrane permeant endoplasinic reticulum-specific luminescent reagents (Molecular Probes, Inc.) are used to label the endoplasmic reticulum of living and fixed cells. These reagents include short chain carbocyanine dyes (e.g., DiOC₆ and DiOC₃), long chain carbocyanine dyes (e.g., DilC₁₆ and DilC₁₈) and luminescently labeled lectins such as concanavalin A.

In a second embodiment, antibodies against endoplasmic reticulurn antigens (Sigma Chemical Co.; Molecular Probes, Inc.; Caltag Antibody Co.) are used to label endoplasmic reticulum components that are localized in specific endoplasmic reticulum domains. Examples of these components are the macromolecules involved in the fatty acid elongation systems, glucose phosphatase, and HMG CoA-reductase.

In a third embodiment, protein chimeras comprising a endoplasmic reticulum protein genetically fused to an intrinsically luminescent protein such as the green fluorescent protein, or mutants thereof, are used to label the endoplasmic reticulum domain. Examples of these components are the macromolecules involved in the fatty acid elongation systems, glucose-6-phosphatase, and HMG CoA-reductase.

Golgi Labeling

In one embodiment, membrane permeant Golgi-specific luminescent reagents (Molecular Probes, Inc.) are used to label the Golgi of living and fixed cells. These reagents include luminescently labeled macromolecules such as wheat germ agglutinin and Brefeldin A as well as luminescently labeled ceramide.

In a second embodiment, antibodies against Golgi antigens (Sigma Chemical Co.; Molecular Probes, Inc.; Caltag Antibody Co.) are used to label Golgi components that are localized in specific Golgi domains. Examples of these components are Nacetylglucosamine phosphotransferase, Golgi-specific phosphodiesterase, and mannose-6-phosphate receptor protein.

In a third embodiment, protein chimeras comprising a Golgi protein genetically fused to an intrinsically luminescent protein such as the green fluorescent protein, or mutants thereof, are used to label the Golgi domain. Examples of these components are N-acetylglucosamine phosphotransferase, Golgi-specific phosphodiesterase, and mannose-6-phosphate receptor protein.

While many of the examples provided herein involve the measurement of single cellular processes, for certain embodiments, multiple parameter high-content screens can be produced by combining several single parameter screens into a multiparameter high-content screen, in which several stains and labels are used to observe several cellular components simultaneously, or by adding cellular parameters to any existing high-content screen. Furthermore, while each example is described as being based on either live or fixed cells, each high-content screen can be designed to be used with both live and fixed cells.

Those skilled in the art will recognize a wide variety of distinct screens that can be developed based on the disclosure provided herein. There continues to be a large and growing list of known biochemical and molecular processes in cells that involve translocations or reorganizations of specific components within cells. The signaling pathway from the cell surface to target sites within the cell involves the translocation of plasma membrane-associated proteins to the cytoplasm. For example, it is known that one of the src family of protein tyrosine kinases, pp6Oc-src (Walker et al (1993), J. Biol. Chem. 268:19552-19558) translocates from the plasma membrane to the cytoplasm upon stimulation of fibroblasts with platelet-derived growth factor (PDGF). Additionally, the targets for screening can themselves be converted into fluorescence-based reagents that report molecular changes including ligand-binding and posttranslocational modifications.

Next, in step 4, the cells are processed through one of the many biological assays well-known to those of skill in the art. Typically an assay experiment is accomplished by subjecting a suspension containing the cells cultivated in step 2 to a perturbation (e.g, using a test-compound reagent) that allow such cells to continue growing or change the way the cells are growing, and adding at a later time the imaging reagent (e.g., a stain or the like) obtained in step 3 that interacts with the desired structures or targets inside such cells. At a given time (depending on specific cell line and stain being used), images of the cells are obtained with an optical system suitable for visualizing the location of the stained material in the cells. Optionally, multiple images can be obtained over multiple times in order to assess the temporal behavior of the cells, or cells in different locations in the assay container (e.g. adhered versus in solution).

For example, the same, living, cell culture can be imaged multiple times during the same assay experiment. Or a series of assay experiments can be conducted with the same cell type and imaging reagents (e.g., labels and same concentration of the same test-compound if a compound is used) where the cells are fixed (killing them) at a series of times and imaged to get images of the response at different times. A series of assay experiments can also be conducted in which the same cell type and imaging reagents (and same time) are run with a series of different concentrations of the same test-compound to evaluate and fingerprint the compound at a variety of different concentrations. Accordingly, each “compound fingerprint” for one cell type and imaging reagent (or mixture of imaging reagents) can comprise of a series of experiments with that cell type and imaging reagent(s) in which each of a series of concentrations of the compound is measured at each of a series of times. The features from each image from each assay experiment of a compound at one time and concentration is subtracted from the features from the ‘reference’ assay experiment of the same cell type and imaging reagents (e.g., at that time) that were not treated by the compound. Either a fingerprint or signature of the entire matrix of time and concentration, or a fingerprint of each experiment of the compound at that time and concentration, can be analyzed by the methods provided herein as a single fingerprint of the compound.

For example, in one embodiment, the recording of images can be made at a single point in time after the application of the perturbation, such as with a test-compound. In another embodiment, the recording could be made at two points in time, one point being before, and the other point being after the application of the influence. In another embodiment, the recording of images can be performed at a series of points in time, in which the application of the perturbation occurs at some time before, on or after the first time point in the series of recordings, the recording being performed with a predetermined time spacing of, e.g., from 0.1 seconds to 1 hour interval, or from 1 to 60 second intervals, or from 5 to 30 second intervals, or from 1 to 10 second intervals; such as every 1 second, every 5, 10, 15, 20, 25, 30 seconds, or the like. The recording of images can be performed over a time span of from 1 second to 24 or more hours, such as from 10 seconds to 12 hours, or from 10 seconds to one hour, or from 60 seconds to 30 minutes, or the like.

Because many assay experiments must be run reproducibly, the assay procedure may be run using automated equipment. An assay experiment is defined to be the process of running an assay and collecting a result. Typically the images will be collected digitally. Systems to perform cellular assay experiments in which cellular images are collected as an assay result are available. See, for instance, U.S. Pat. No. 5,989,835, System for Cell-Based Screening, of Cellomics; and U.S. Pat. Nos. 4,741,043; 6,026,174; 5,983,237; 5,579,471; 6,103,479; 5,548,661; 5,828,776; 5,852,823; or the like; and PCT WO 99/39184, PCT WO 00/17643, and the like; each of which is incorporated herein by reference in their entirety.

The set or sets of images are obtained from each assay experiment conducted in step 4, and each cell within each image are archived to form the reference images. In FIG. 1, block 5 represents the archived image(s). As a matter of convenience, these images can optionally be stored (e.g., digitally) for later use to repeat the computer analysis of the images, as set forth below.

With reference to FIG. 1, step 6, a computer processes the images of the cells with software in order to digitally Identify and quantify various features in the cells cultivated in step 2 and stained in step 4. The image processing software can identify specific image features that result from the assay created with a cell line or stain. Alternatively, and in a particular embodiment, the image processing software runs a standard suite of image feature detection algorithms regardless of the expected assay change. For example, and without limitation, well-known image processing software can run some or all of the following image analyses on each image collected from normal or treated cells:

Global image statistics such as area such as total gray value, optical integrated density (OD), etc.

Image analysis can be conducted on single cells identified in the image(s), including the analysis of size and shape such as: perimeter, centroid X and Y, Z-position, width, length and height, orientation, breadth, fiber length, fiber breadth, inner radius, outer radius, mean radius, average gray value, total gray value, optical integrated density (OD), intensity center location, radial dispersion, texture difference moment, OD variance, and others

Cell population statistics can be collected from each assay image, including: cell count, cell density, histogram of different identifiable states, population diversity, and statistics of any single-cell features described above, and others

Temporal statistics can be collected from each assay that would yield insight into the change of any image feature over time.

The result of the image processing software is stored and manipulated in a data set referred to herein as image descriptors. The result of the entire set of image processing algorithms then forms the image descriptors, which will be a characteristic fingerprint of the assay image. Block 7, the output of step 6, represents the image descriptors from an assay experiment. Typically many experiments are run for each particular assay type and the image descriptors averaged so that the resulting descriptors reflect the average image observed for the assay type.

B. Creating One Reference Assay Image Change for One Compound

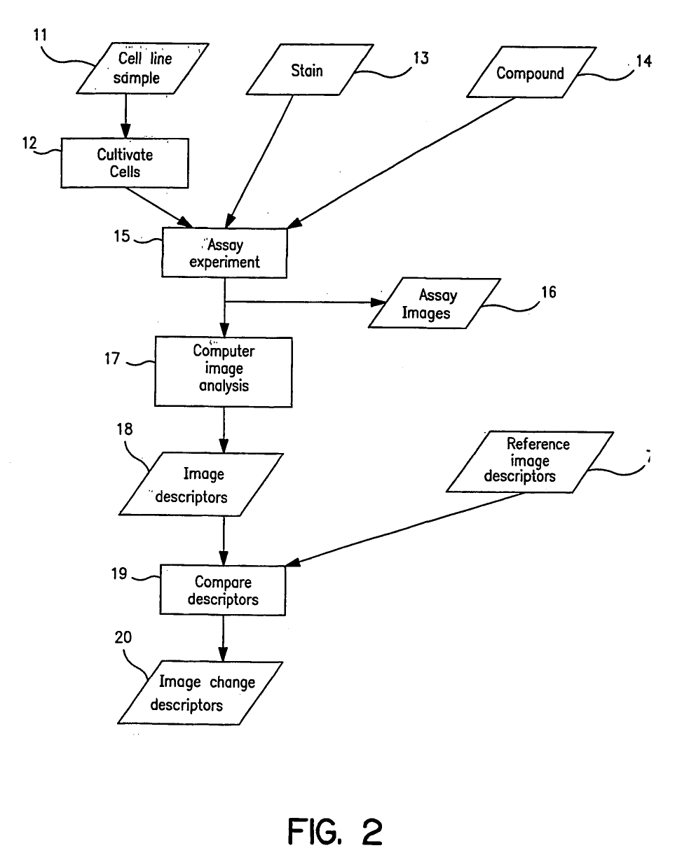

Once the standard, reference image(s) of a particular cell line in an assay is characterized as described in Section A above, then changes to such cell line are induced by treating or perturbating the cell line with, for example, a biologically active compound such as a chemical used as a drug, or the like. The changes in the biological activity of the cell line as a result of such perturbation are observed through changes to the images of the cells. FIG. 2 is a flow chart illustrating an exemplary set of processes performed, either manually and/or automatically using well-known high throughput assay systems and devices, to assess the change in images of cells in an assay that results from the effect of each test-compound selected.

The first step is to culture a batch of the cells to be used in the assay under substantially the same culture conditions, in particular embodiments exactly the same culture conditions, used in step 1 for this assay. In FIG. 2, steps 11, 12, and 13 are exactly analogous to steps 1, 2 and 3 of the procedure set forth in Section A. Again, tight control of the cell cultivation conditions ensures that the cell line will behave exactly the same way in this assay as in the reference image assay described above. Next, in step 14, a perturbation is selected (e.g., a biologically active compound) that may change the behavior of the cells in that assay and also change the new assay images of those cells.

In step 15, the cell culture, imaging reagent (e.g., a stain), and the perturbation (e.g., a test-compound) are processed in an assay experiment. In a particular embodiment, this assay must be accomplished under exactly the same conditions and with the same procedure as carried out in step 4 for the cell line chosen (typically using the same experimental equipment and/or at the same time in parallel), with the exception that to each cell culture of step 15 is additionally introduced the perturbation (e.g., compound) of which biological effect on the cell line is to be characterized. At a given time, images of the cells in each experiment are obtained with the same assay optical system used in the initial assay described above and in FIG. 1. Optionally in this step, several experiments may be run with different amounts of the same perturbation, in order to determine the relationship between the quantity of the perturbation (e.g., concentration of the compound) and it's effect on the cells. Also, optionally, multiple images can be created over time and/or space in order to assess the change in temporal behavior and/or spatial distribution of the cells in the cell line. The set of assay images from this experiment, and each cell within each image, is represented by step 16 in FIG. 2. The images of the cells are processed by the computer with the same image processing technique used in step 6 for this type of assay (i.e., for each combination of cell line and stain). The results of the computer analyses of the assay images are compiled into a data set (e.g., a digital data set) that serves as the descriptors of such assay images (block 18).

In order to assess the change in biological activity caused by the introduction of the chosen perturbation to the assay, the reference images for this assay must be compared to the images from the assay experiment run with the same perturbation (e.g., same compound). In step 19 the computer compares the reference image descriptors (obtained with the procedure set forth in Section A) with the assay image descriptors (the output of step 17). See block 18 of FIG. 2. The computer will establish a description of the change in images based on a comparison of the descriptors; “otherwise known as assay response image changes.” By way of example and without limitation, the description for the image change may take the form of a descriptor vector, each element of which may be calculated as the difference in the value of the corresponding elements in each image's descriptors. The changes in the descriptors obtained from the image processing algorithms from the analysis of the images, is the data set containing the descriptors of the reference image changes, and serves as the identifying pattern(s) of the biological effect of the chosen perturbation (e.g. compound) on the chosen cell line, as visualized by the chosen imaging reagent (e.g., stain). This data is indicated as block 20 in FIG. 2.

C. Creating a Library of Reference Image Changes for One Assay for a Multiplicity of Compounds

Many reference image changes in a given cell line are created using many different perturbations (e.g., different test-compounds). In this method, p different biologically active perturbations are selected, D, with each perturbation denoted D_{z} and where z runs from 1 to p. There are tens of thousands of known biologically active compounds. The difference in assay responses caused by each of the perturbations (i.e., reference image changes) are fingerprints, or identifying patterns, of the biological mechanisms affected by each of the p perturbations. In a particular embodiment, the p perturbations are chosen so that they cause changes in the widest possible range of different cellular processes affecting a wide range of different biological mechanisms within the cell of a cell line.

Although the biological mechanism affected by a particular test-compound is not required to be known for use in the methods described herein, in particular embodiments, known biologically active test-compounds affecting known biological mechanisms are employed. Exemplary biologically active test-compounds known to affect a diverse set of particular biological mechanisms are very well-known in the art as described, for example, in THE MERCK INDEX, An Encyclopedia of Chemicals, Drugs, And Biologicals, Eleventh Edition, 1989, Merck & Co., Inc., Rahway, N.J., or the like; and THE PHARMACOLOGICAL BASICS OF THERAPEUTICS, Ninth Edition, Hardman et al., (each of which are incorporated herein by reference in its entirety). For example, an exemplary set of 640 pharmacologically active compounds are sold as a set from Sigma-Aldrich and often used as a compound panel for assay validation and high throughput screening.

To create the desired library, it is necessary to repeat the process described above in reference to FIG. 2 for each of p perturbations. Different amounts of each perturbation may be tested with each chosen cell line and stain in several assay experiments, so that the effect of concentration on the observed cellular change can be assessed. The result of this process is p descriptors of reference image changes. Each of the p descriptors of represents the observable changes in this assay (a single cell line and stain) due to the biological activity of each of the p perturbations. Each of the p descriptors itself may, optionally, be a set of descriptors where each of the set may represent the effect of different concentrations of the perturbation and/or the effect on cells in different locations and/or at different times and/or in different life cycle stages.

D. Creating a Library of Reference Image Changes For a Multiplicity of Assays and a Multiplicity of Perturbations

The above described process is repeated to create a large set of observations of changes in the normal biological functionality caused by p perturbations (e.g., test-compounds) in a large number of different assays. Each assay is defined as the use of one stain to visualize the biological activity of one type of cells. To create the desired library it is necessary to choose n different types of cells, C, with each cell line denoted Cₓ where x ranges from 1 to n. Next, it is necessary to choose m different stains, S, with each stain denoted S_{y} where y ranges' from 1 to m. The number of assays is the product of n and m. In a particular embodiment, the n cell lines and m stains are chosen so that they allow observation of a wide range of different biological activities from a wide range of different biological mechanisms. For example, there are about 4000 different cultivatable cell lines and about 2000 different intercellular stains specific to different internal parts of the cells of these cell lines.

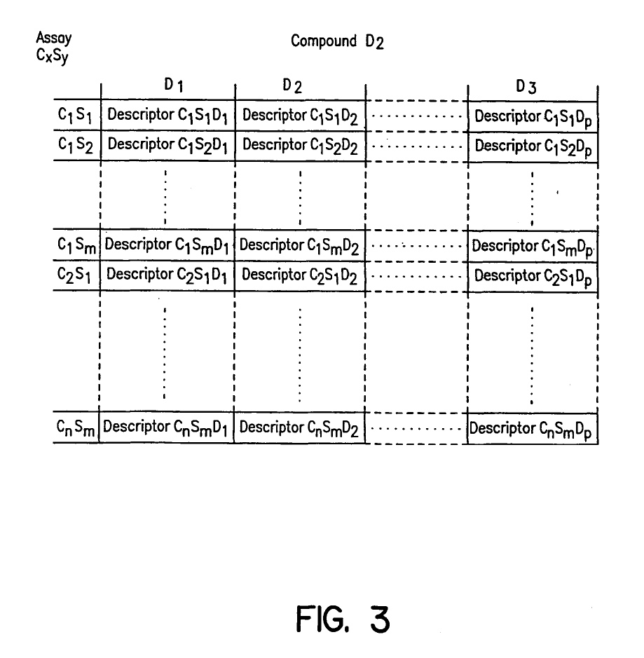

The process is carried out as follows. First, the process described in reference to FIG. 1 is carried out for each of the n x m assays, creating reference image descriptors for each assay that reflect the features of the image from normally functioning cells in an assay. Next, for each of the n x m assays, it is necessary to perform the process described in Section C above for each of p perturbations, creating a description of the p observable changes caused by the p perturbations in each of the assays. The library of reference image changes, is the change in assay response caused by each of p perturbations in each of n×m assays, or n x m x p descriptors of biological changes. Again, each of the p descriptors itself may optionally be a set of descriptors where each of the set may represent the effect of different concentrations of the perturbation and/or the effect on cells in different locations and/or at different times and/or in different life cycle stages. FIG. 3 is an exemplary matrix representation of the library of descriptors of reference image changes. Each of the assays defines a row in the matrix and each of the tested perturbations (in this case compounds) represents a column in the matrix. In this method, library of reference image changes is represented in the computer by a set of descriptors.

E. Analysis of a Library of Reference Image Changes for Patterns That Correspond to Individual Cellular Biological Mechanisms.

It is likely that a biologically active cellular perturbation, such as a test-compound, will affect several biological mechanisms in many different cells simultaneously. The multiple affects of an active perturbation may be visible as reference image changes in different assays. Multiple mechanisms of a bioactive cellular perturbation can also be exhibited in a single assay. In accordance with the methods provided herein, statistical methods are applied to identify components of specific assay responses that result from the perturbation of individual biological mechanisms.

1.) For example, drug A and drug B may have very different disease related targets, but can both cause the same side effect through the interaction with a third metabolic pathway. With the above-described library, one can observe (via the reference image changes) drug A's interaction with the desired target in a metabolic pathway in one assay, and drug B's desired biological activity in an another assay and, separately, the side effect of both drugs in a third assay. Data mining of the above-described library will identify the similar response of drug A and drug B in the third assay as due to the affect of both compounds on a similar biological pathway.

Multiple mechanisms of a bioactive compound can also be exhibited in a single assay. For example, compounds I and J, which could again be drugs used against different therapeutic targets, both could contain an aromatic ring in their chemical structure. In a K assay of both compounds, a change in the chosen cells due to the aromatic ring found in both compounds is observed. A change is also observed in the cells when J is assayed that results from J's interaction with one of the metabolisms visualizable in assay K. In certain embodiments, the response of most assays will be due to the complex effect on several metabolisms in the cells used for that assay.

In certain embodiments, the observed changes in each assay response image for any compound is due to the sum of all changes in the assayed cell's mechanism that can be visualized with a particular stain. Thus, the observed reference image changes descriptor reflects contributions from changes in a multiplicity of mechanisms. In other words, in these embodiments, the reference image change descriptor for a compound is not expected to be the result of the affect of that compound on a single metabolic pathway. The individual metabolic pathways affected by each of the compounds can be ascertained by finding and grouping patterns of image change descriptors between the p compounds. For example, in the paragraph above, pattern recognition techniques can be used to identify that the signatures of compounds I and J in the K assay share a sub-pattern due to a shared effect, but differ by the additional effect of compound J. Each assay response descriptor and the n x m assay response descriptors, collectively for each of the compounds can be subset (e.g., using well-known clustering methodology) into sub-patterns of assay responses (the sum of all sub-patterns then making up the observed pattern or patterns). In other words, the image response pattern in the fingerprint vector results from the sum of a number of sub-patterns, each of which is identified separately, where the individual sub-patterns are superimposed to create the image response pattern. These sub-patterns may correspond to individual biological activities or a subset of all the biological activity mechanisms affected by a compound or group of compounds or by a chemical substructure of the compounds. Exemplary clustering methods for use herein include one or more of “fuzzy clustering” and “multi-domain clustering”, and the like.

The end result of the data mining techniques applied to the library of reference image change descriptors is a pattern or sub-pattern of changes in the descriptors, seen in one or more assays of one or more compounds, for each of the cellular biological pathways that can be affected. These changes, seen in the corresponding assays, then become the signature of any unknown compound or cellular change that affects that pathway in the same way.

Accordingly, methods are provided herein of identifying multiple mechanisms of bioactive compounds, comprising culturing a first reference cell under reproducible conditions; processing the first reference cell through a multiplicity of assay experiments in the absence of a perturbation; collecting one or more images of the first reference cell to detect a first cell assay response to the respective assays; culturing a second test-cell under the reproducible conditions of step a), wherein the first reference cell and the second test-cell are the same cell species; processing the second test-cell through the same multiplicity of assay experiments of step b) in the presence of a perturbation; collecting one or more images of the second test-cell to detect a second test-cell assay response to the respective perturbation; comparing the one or more images obtained of the first reference cell to the one or more images obtained of the second test-cell to identify assay response image changes between the first reference cell and the second test-cell, wherein the assay response image changes correspond to a fingerprint of assay responses caused by the perturbation repeating steps a) through g); with a multiplicity of perturbations; and identifying shared patterns of assay response image changes between the multiplicity of perturbations and identifying within the shared patterns, a specific sub-pattern of assay response image changes, wherein the sub-pattern of assay response image changes corresponds to an individual biological mechanism or a subset of all biological mechanisms affected by the subgroup of perturbations. The specific sub-pattern of assay response image changes can be identified using one or more of the well-known statistical clustering techniques, such as fuzzy-clustering, multi-domain clustering, and the like.

2.) A Specific Example of Identifying Multiple Mechanisms of Bioactive Compounds.

Aspirin (acetyl salicylic acid) has been used since 1899 for relieving pain and fever. This drug and other, more modern non-steroidal anti-inflammatory drugs (NSAIDs) like Ibuprofen, Naproxen and others, constitute one of the largest groups of pharmaceuticals. Although NSAIDs have been extraordinarily useful in controlling pain associated with musculoskeletal disorder and inflammation as well as a variety of other conditions, it is now appreciated that their use is associated, with significant side effects, primarily because of gastrointestinal toxicity, but also because of renal dysfunction and cardiac failure. Until 10 years ago, it was generally accepted that NSAIDs acted by reducing prostaglandin synthesis through inhibition of cyclooxygenase (COX). It has recently been found that there are actually two forms of the COX enzyme; COX-1, which is necessary to maintain overall health; and COX-2, which is linked to inflammation and tumor formation. This realization led to the development of drugs that are selective COX-2 inhibitors like Rofecoxib and Celecoxib.

As a result, one or more assays of several drugs in the broad class of NSAIDs will yield responses due to the range of specific activities. Some drug compounds like Aspirin and Ibuprofen will have an assay response resulting in their inhibition of both forms of COX while other drug compounds like Rofecoxib and Celecoxib will have an assay response that results from their selective inhibition of just the COX-2 isoform. These multiple effects may result in one complex assay response or result in different assay responses. For example, there may be one assay with one change that is specific to COX-1 inhibition and a separate assay with a distinct change that is specific to COX-2 inhibition. In this special case of two separate assays, the COX inhibitors would cause a response in both assays and the COX-2 inhibitors would only cause a response in the latter assay and so these two groups of compounds could be separated easily based on their assay response. However as a general rule, the response of compounds in most assays will be due to their complex effect on several metabolisms in the cells used for that assay. For example, even if separate assays were created for COX-1 and COX-2 inhibition, with some cell types (for example, hepatic cells) used in a COX-2 assay, Rofecoxib and Celecoxib would have different responses because Celecoxib inhibits CP450 (CYP2C9) enzymes and Rofecoxib does not.

In general, it is likely that a biologically active compound will affect several biological mechanisms simultaneously in the cells used for an assay. These multiple effects may result in a complex assay response if the images of the cells change in a way that reflects the multiple effects. The observed changes in each assay response image for any compound is due to the sum of all changes in the assayed cell's mechanism that can be visualized with a particular stain. Thus, the observed reference image changes descriptor reflects contributions from changes in a multiplicity of mechanisms. In other words, in this particular embodiment, the reference image change descriptor for a compound is not the result of the affect of that compound on a single metabolic pathway. The individual metabolic pathways affected by each of the compounds can be ascertained by finding and grouping patterns of image change descriptors between the p compounds.

To continue the NSAIDs example from the paragraphs above, pattern recognition techniques can be used to identify that the assay responses of the general and specific COX-2 inhibitors in an assay (or assays) have a similar pattern due to a shared inhibition of COX-2 effect, but differ by a sub-pattern that results from the additional effect that some of the compounds also inhibit COX-1. Each assay response descriptor and the n x m assay response descriptors, collectively for each of the compounds can be subset into sub-patterns of assay responses (the sum of all sub-patterns then making up the observed pattern or patterns). These sub-patterns may correspond to individual biological activities or a subset of all the biological activity mechanisms exhibited by a compound or group of compounds or by a chemical substructure of the compounds.

In accordance with the methods provided herein, statistical analysis techniques are applied to a database of image change descriptors generated by assaying a set of compounds that allows the multiple patterns in each image response to be separated and classified and optionally assigned to the specific mechanism of cellular biological activity. The end result of the statistical data mining techniques applied to the library of reference image change descriptors is a pattern or sub-pattern of changes in the descriptors, seen in one or more assays of one or more compounds, for each of the cellular biological pathways that can be affected. These changes, seen in the corresponding assays, then become the signature of any unknown compound or cellular change that affects that pathway in the same way.

The statistical analysis methodology applied herein to separate sub-patterns of assay response by unique mechanism specifically allows for the multiple classification of compounds into groups that share each of the multiple mechanisms. For example, traditional clustering methods are used to partition a data set into clusters or classes, where similar data are assigned to the same cluster whereas dissimilar data should belong to different clusters. In one particular embodiment, however, there is no sharp boundary between clusters by mechanism (for example compounds will have differerit degrees of an affect on a mechanism), such that fuzzy clustering can be advantageously utilized. In fuzzy clustering, membership degrees between zero and one are used instead of crisp assignments of the data to unique clusters. In addition, in this embodiment, there is no unique cluster that defines a biological mechanism to which a compound will belong. Rather the compound is assigned membership in each cluster that defines a separate biological effect that is observed in the assay responses.

Multi-domain clustering is a statistical data mining approach that partitions data sets into clusters where different similarities (e.g., different sub-patterns within the pattern created by the image response descriptors) are identified and data is assigned to each cluster in which it shares the defined similarity. Clusters where “different similarities” are identified can also be referred to as unique combinations of a few of the image features from the complete vector of image features. Accordingly, methods are provided herein that use statistical, fuzzy clustering and multi-domain clustering techniques for identifying and classifying the unique assay responses due to separate biological activities of the tested compounds (or other cellular perturbations) when the multiple biological effects of the perturbations in combination result in the pattern observed in the cellular image response from the assay. A unique advantage of this approach is the performance of clustering to determine separate biological activity mechanisms from the image response data by using fuzzy clustering to establish degree of cluster membership and/or multi-domain clustering to establish multiple cluster memberships.

Fuzzy clustering is a statistical technique for clustering in a fashion that allows for “degree of membership” to a cluster. This technique has been applied to a number of classification applications including, image recognition, data analysis and rule generation. A number of fuzzy clustering techniques like the fuzzy c-means, the Gustafson-Kessel and the Gath-and-Geva algorithm for classification problems have been developed and are well known in the literature. One thorough review of this area and applications of fuzzy clustering is available in the book Fuzzy Cluster Analysis, Wiley (1999) ISBN O-471-98864-2, incorporated herein by reference in its entirety. Several established fuzzy clustering techniques are particularly useful in the methods provided herein, including the fuzzy c-means and the Gath-and-Geva algorithm.

Multi-domain clustering is not a standard field of statistical analysis. Rather specific and unique multi-domain clustering methods are typically developed for specific applications. For example, a number of multi-domain clustering techniques have been developed for protein homology searching. With these techniques, protein sequences can be assigned to multiple clusters based on homology between separate amino acid sequence regions in that protein or separate parts of the 3-dimensional structure of the protein. A separate example of the development of a unique multi-domain clustering application is the use of interaction maps to study and establish multiple protein-protein interactions (“Generating Protein Interaction Maps from Incomplete Data: Application to Fold Assignment” M. Lappe et al, Bioinformatics Vol 1, no. 1. 2001, pp 1-9).

In accordance with the methods provided herein, multi-domain clustering is applied to the complex fingerprint of cellular assay image responses that result from compound testing in one or more assays. For example, one comprehensive approach for multi-domain clustering is the execution of a comprehensive set of fuzzy clustering analyses on every possible subset of data in the database of assay responses of a set of compounds. In this embodiment, consider image change responses from a set of 10 compounds assayed in each of 10 assays. In this embodiment, each assay is a unique combination of a cell line and labels that generate a unique image and, for example, a compound's image response is the data produced for that compound, which may include multiple responses over a range of concentrations and times.

A standard fuzzy clustering computation can, for example, use the Gath- and Geva algorithm to cluster the data for each compound in one assay to look for similarities in assay response. Typically, the multi-domain clustering method is undertaken by clustering every possible subset of the compounds, e.g, by clustering in a separate application of the Gath-and-Geva algorithm each possible pair of compounds, each possible unique set of three compounds, each possible unique set of four compounds, each possible unique set of five compounds, each possible unique set of six compounds, each possible unique set of seven compounds, each possible unique set of eight compounds, and each possible unique set of 9 compounds along with the full set of 10 compounds. At most, this would entail 10! (ten factorial) clustering computations run separately. In addition, this multi-domain clustering analysis can repeatedly perform the clustering computations on the set of 10! unique combinations of compounds, with each repeat of the set of clustering analyses performed with a unique combination of assay results. With 10 different assays of a compound, there are again 10! possible unique combinations of assay results for each compound. Thus, the 10! unique clustering computations for the unique combinations of compounds in the compound set can be run for each of the assay responses separately and then again for the 45 different ways of combining two assay responses into the response for that compound that is used for clustering, and then separately again for the unique combinations of three assay responses combined into the response for each compound, and so on. Complete exploration of the 10 compound by 10 assay space with standard fuzzy clustering would entail 10! X 10! clustering analyses, although some of these analyses will not make sense, such as clustering the response of just two compounds, and the like. In addition, sub-patterns in the response fingerprint from each assay can be analyzed by clustering with every possible combination of the elements of the fingerprint. For example, in a fingerprint with 100 individual features or attributes of an image change, each of the unique compound and assay clustering computations described in the previous experiment can be repeated with a unique subset (at most 100 factorial) of fingerprint elements.

In this exemplary multi-domain clustering analysis, the results of all the clustering computations are analyzed to identify significant changes in membership associations between compounds. For example, there may be a large set of clustering computations where a group of five of the ten compounds fall in the same cluster if their data is included in the clustering computation. However, in the results of another set of clustering computations the group of compounds may be significantly and reproducibly assigned membership in separate clusters. This situation, identified from the results of all of the clustering computations, is then a candidate for multi-domain classification where the group of five compounds may be similar in one assay response area and different in another.

Comprehensive, combinatorial clustering of the complete library of assay responses is a computationally huge task. In this application, expert judgements can be used to minimize the combinations investigated by clustering. For example, the minimum expected set of compounds that are similar to one another can be used to avoid clustering small numbers of compounds, like sets of two and three compounds. Additionally, a reduced set of image features in the image response fingerprint can be explored for the effect on changing groupings rather a complete factorial exploration of every element.

There are additional ways in which the n x m x p reference image changes and their descriptors can be further subdivided and analyzed to facilitate the fuzzy and multi-domain clustering investigations of separate biological activity mechanisms using the library of assay image responses. For example, if the different stages of a cell's life cycle are considered as a separate cellular system, then the change of that assay to a compound can be divided into the effect on dividing cells, the effect on quiescent cells, and the relative population of cells in different life cycle stages. In another example, the p compounds can be further described by the chemical structural features of the compound, allowing the assay responses to be investigated by chemical feature.

F. Investigating Activity Mechanisms of an Unknown Perturbation Using the Complete Library

1. Hypothesizing the Similarity of Biological Activities From the Similarity of Assay Response Descriptors

By running the process described in Section B, above, the biological activity mechanisms of an unknown cellular perturbation (e.g., a test-compound) can be investigated by assaying the perturbation in each of the n x m assays for a subset of the assays) used to create the above described library. For each of the n x m assays, the response descriptors from the unknown perturbation are compared with the p descriptors (and patterns found in the p descriptors) observed for the p known biologically active perturbations. Similarity between the image change descriptors observed for the unknown perturbation and one or more of the p perturbations is evidence of similarity of biological activities. Making this, comparison of through the descriptors or descriptor patterns does not require identifying specific biological mechanisms that give rise to the descriptors or descriptor patterns in the library. Rather the similarity of the descriptors or descriptor patterns alone are used to make the inference that one or more of the biological activity mechanisms are similar.

2. Identifying Known Biological Activity Mechanisms

In a particular embodiment, the biologically active perturbations to be assayed for the creation of the library are compounds chosen based on a large amount of publicly available knowledge about the biochemistry underlying their biological activities. For example, these compounds may have been used or investigated as pharmaceutical drugs. In this example, drugs and drug-like compounds are preferentially chosen for inclusion in the set of p compounds because the biological activities of each compound have been extensively investigated and results published. These biological activities typically include both the biological mechanism(s) efficacy against a disease-related targets) as well as other biological mechanisms caused by the compound (e.g. side effects, toxicity, etc). When the biological activity mechanisms of a compound are known the mechanisms elucidated for the known compounds can be associated with specific observed assay responses or patterns or patterns or sub-patterns of assay responses.

When patterns or sub-patterns of assay responses have been assigned to the affected biological mechanisms that cause them, then matching an assay response pattern, caused by an unknown compound allows direct identification of the biological activity mechanism this unknown compound affects. This method can be used to identify any biological activity mechanism, both desirable and undesirable. For example, when one or more of the p known compounds are toxic, the similarity in the assay responses caused by the investigated compound and the pattern observed for the known toxic compound is predictive evidence that the investigated compound(s) may have the same toxicity by the same activity mechanism. Thus, the methodology provided herein sets forth a mechanism to predict any in vivo biological activity through the similarity in the pattern of assay responses of the investigated compound and known compounds.

3. Discovering Biological Activity Mechanisms

When a known biological activity mechanism cannot be assigned to an observed assay response or pattern or sub-pattern in the responses, this method can be used to discover such assignments. The n x m x p assay response descriptors can be investigated for similarity, for example with statistical similarity clustering techniques. If similarity is discovered among the patterns in the library, the descriptor similarity can be mapped back to the images collected and the compounds that caused the patterns can be investigated to determine if they share similar biological activity mechanisms. In this manner, unknown biological activity mechanisms can be discovered and investigated.

4. Identifying Disease-Related Targets

The similarity in the assay responses between a compound being investigated and the known compounds used to create the library can be used as evidence that the unknown compound may have utility as a drug lead against the disease state and/or therapeutic target that is addressed by the known compounds. Thus, provided herein is a method to identify which disease state or therapeutic target may potentially be treated by biologically active compounds.

G. Investigating the Role of a Gene Biological Activity

1. Identifying the Biological Mechanisms Affected by a Gene

The biological activity mechanisms in which a gene and/or gene product play a role can be assessed by observing the change in cell response to a panel of assays that results from modifying the gene expression of the gene in a cell line. For example, in order to assess the biological activity of a gene, a modified cell line is created in which the expression of the protein corresponding to the gene being investigated is altered, such as enhanced or suppressed. This modified cell line is then used in m x p assays in which the unmodified cell line was a component (with all stains and with all biologically active reference perturbations). The difference in the m x p assay responses between the modified and unmodified cell line is observed. The difference in responses of the assays to some of the perturbations when compared to the original assays with unmodified cells is evidence that the gene and/or gene product is involved in one or more of the biological mechanisms affected by the perturbations.

One specific advantage of this method is that it enables the identification of the function of genes the manipulation of which does not cause a phenotypic change in the cell line. By assaying p perturbations against the cell line, this method forces changes in the biological activity of the cell caused by the perturbations. Thus the function of genes in response to perturbations in the normal metabolism caused by the assay can be identified.

As another example, in order to assess the biological activity of a gene, a specific type of cell (cell line) can be used in series of assays of a panel of biologically active compounds. In one embodiment, the commercially available LOPAC set of 640 compounds from Sigma-Aldrich can be used as the compound panel. In other embodiments, a range of compounds is selected for inclusion in the panel that has a diverse range of known biological effects. Each of the compounds in the panel will cause the cells to respond in the assay in a characteristic manner, which is captured by the image change assay response. The result of assaying each compound yields the characteristic assay responses of that cell type to each compound in the panel, which can be placed in a database or otherwise stored. Each of these assays for each of these compounds is used as a method of establishing the characteristic response of the specific cell type to treatment with these compounds.

In order to explore the function of a gene, the expression of the gene or gene product can be modified in the cell line that has been subject to the standard assays described above. This altered modified cell line can be used in another set of assays of the same standard panel of compound that was previously used to characterize the response of the unmodified cell line. Each of the compounds in the panel will cause the modified cells to undergo a biological response, which is captured by the image change assay response for that compound. The result is a set of assay responses of the modified cell type to each of the compounds in the panel. The function of the gene that was the subject of the modification is then investigated by comparing the response of the modified cell line to the response of the unmodified cell line in each assay of each compound in the panel. Individual compounds that cause a different response in the modified cell line compared to the response in the unmodified cell line are affecting a biological mechanism that has been changed as a result of the gene modification.

In particular embodiments where compounds included in the panel have well known biological activities, useful information from the process described above will be generated. For example, numerous biologically active compounds can be selected for inclusion in the panel that have been previously studied. Compounds that have been used as drugs are good candidates for inclusion in the panel because their mechanism of biological activity are typically extensively studied, well known, and published in the technical literature. Using compounds with well known biological activity in the panel facilitates the identification of the gene function when the response of the compound changes as a result of the modification of that gene. First, however, the identification of which of the potentially multiple effects of the compound were modified as a result of gene modification will be ascertained.

In particular embodiments where gene function is analyzed through the identification of compounds in the panel that elicit a different cellular response as a result of the gene modification (as set forth above), useful information will be generated from the multiplicity of compounds whose assay responses change as a result of the gene modification. For example, if the assay response of several compounds each change as a result of gene modification and those compounds are known through other studies to have a similar biological activity, it can be inferred that it is the similar biological activity that was affected by the gene modification. For example, if each of the compounds in the panel that have a different assay response in the modified cell line are known to reduce prostaglandin synthesis through inhibition of cyclooxygenase and have no other similarities known in the literature, then it can be inferred that the gene plays a role in this or a related biological mechanism. The compound panel used in these embodiments can be designed to have diverse and well-known biological activity that also provides redundancies or similarities in biological activity that will identify functions.

The methods described herein for investigating gene function will provide substantial advantages over the current state of the art in gene modification studies. In widely used gene modification studies (e.g. gene knockout studies), a typical procedure involves gene modification of an organism (for example a cell line or a microorganism or a mouse) followed by close inspection of the modified organism for an obvious, observable change that resulted from modification. Typically in these studies there are at least three possible outcomes. The first possible outcome is the absence of a viable organism when the absence of the gene does not allow the organism to live. A second outcome is the production of a modified organism with no observable difference from the corresponding unmodified organism (e.g. a “silent knockout”). The third, desired outcome is the production of a modified organism with an observed difference from the corresponding unmodified organism that can be used as a starting point to identify and study the function of the gene that was modified.

One advantage of the methods of gene function analysis provided herein is the reduction of the incidence of the second outcome described above in which no observable difference is found as a result of modification. In accordance with the methods provided herein, the panel of compounds used to assay the unmodified and modified cell line is designed to force the cells to undergo a biological change. The compounds selected for the panel can be chosen such that they cause a broad range of such changes. When responding to the challenge presented by each of the broad range of compounds in the panel, the modified and unmodified cell lines will be forced to respond by changing a wide range of biological mechanisms. Some of these biological mechanisms may not be normally present in a cell that is not challenged with a compound, which will allow observation of changes in these biological mechanisms that would not be visible if the cell were not forced to respond to the biologically active compound. In other words, unchallenged cells may not exhibit the biological mechanisms in their unchallenged state and so will not allow the changes to the biological mechanisms to be observed.

For the methods provided herein, those of skill in the art will can readily identify and use cell lines that are known to, or suspected to, exhibit biological mechanisms involving the gene of interest. Other methods, such as gene expression profiling, can be used to identify which of the many candidate cell lines that can be used in this method, express the gene to be studied.

2. Associating Multiple Genes Together by their Participation in a Biological Mechanism

A biological mechanism typically has many steps, and typically different genes and gene products are associated with different steps. Affecting the biological mechanism, for example by treating the cell with an active compound, may cause the same observable changers in the cell no matter which step in the activity mechanism is affected. The methods provided herein allow the identification of which different genes play a role in the same biological activity mechanism. In this method, one or more cell lines is genetically modified so that the expression of each gene being investigated is enhanced or suppressed. Each genetically modified cell line is used in the m x p assays (with all stains and with all known active compounds). When two cell lines, each with a different genetic modification, have similar phenotypic changes observed in the m x p assays, there is evidence that the genes that were the subject of modification play a role in the same biological mechanism.

What is claimed is:

1. A method of identifying the biological mechanisms affected by a selected gene, comprising
a) culturing a first reference cell under reproducible conditions;
b) processing the first reference cell through an assay in the presence of a perturbation;
c) collecting one or more images of the first cell to detect a first cell assay response to the respective perturbation;
d) culturing a second cell under the reproducible conditions of step a), wherein the first reference cell and the second test-cell are the same cell species, and the second test-cell is altered to modify the expression of the protein encoded by the selected gene;
e) processing the second test-cell through the assay of step b) in the presence of the same perturbation;
f) collecting one or more images of the second cell to detect a second test-cell assay response to the respective perturbation;
g) comparing the one or more images obtained of the first reference cell to the one or more images obtained of the second altered test-cell to identify assay response image changes between the first reference cell and the second test-cell, wherein the assay response image changes correspond to the biological mechanisms affected by the selected gene.

2. The method of claim 1, further comprising repeating steps a) through f); with a multiplicity of perturbations; and comparing the multiplicity of images obtained of the first reference cell to the multiplicity of images obtained of the second altered test-cell to identify assay response image changes between the first reference cell and the second test-cell, wherein assay response image changes are used to link the biological mechanisms affected by the selected gene with the biological mechanisms affected by the perturbations.

3. The method of claim 1, wherein the perturbation is selected from any one or more of the forces selected from the group consisting of chemical, biological, mechanical, thermal, electromagnetic, gravitational, nuclear, and temporal.

4. The method of claim 3, wherein the perturbation is treatment with a test-compound.

5. The method of claim 4, wherein the test-compound is known to modulate one or more known biological mechanisms.

6. The method of claim 2, wherein the multiplicity of perturbations is treatment of the cells with a multiplicity of test-compounds.

7. The method of claim 6, wherein the multiplicity of test-compounds are each known to modulate one or more known biological mechanisms.

8. The method of claim 1, wherein the first reference cell is labeled with one or more imaging reagents corresponding to the respective assay, and wherein the second test-cell is labeled with the same one or more imaging reagents of step b).

9. The method of claim 1, wherein steps a) through 9) are repeated for a multiplicity of different imaging reagents.

10. The method of claim 8, wherein the one or more imaging reagents are selected from any combination of cellular stains and molecular labels.

11. The method of claim 1, wherein the images are digitally converted to features.

12. The method of claim 2, further comprising correlating the assay responses caused by the test-compounds to the biological mechanisms.

13. The method of claim 1, wherein the expression of the protein encoded by the selected gene is suppressed.

14. The method of claim 13, wherein the expression of the protein encoded by the selected gene is suppressed by knocking out the selected gene.

15. The method of claim 1, wherein the expression of the protein encoded by the selected gene is enhanced.

16. The method of claim 1, wherein a series of images are collected over time to assess the temporal behavior of the first and second cells.

17. The method of claim 16, wherein the images are collected after multiple times, during the same assay experiment.

18. The method of claim 16, wherein the cells are fixed prior to collecting the images.

19. The method of claim 16, wherein the images are collected at different times on different assay experiments of the same cell species.

20. The method of claim 1, wherein the images collected are of different assay experiments of same cell type subject to the same perturbation at different quantities.

21. The method of claim 20, wherein the perturbation is a test-compound administered at different concentrations.

22. The method of claim 1, wherein the images are collected from different locations within the first and second cells.

23. The method of claim 1, wherein the images are collected from different locations within the assay container containing the first and second cells.

24. The method of claim 1, wherein the first and second cells are cell lines.

25. The method of claim 1, wherein the assay response image changes are associated with the respective perturbation and stored in a database.

26. The method of claim 1, further comprising repeating steps a) through f); with a multiplicity of cell types; and comparing the multiplicity of images obtained of the multiplicity of first reference cells to the multiplicity of images obtained of the multiplicity of second altered test-cells to identify assay response image changes between the multiplicity of first reference cells and the multiplicity of second test-cells, wherein assay response image changes correspond to the biological mechanisms affected by the selected gene in the particular cell type in which a change is detected.

27. The method of claim 2, further comprising repeating steps a) through f); with a multiplicity of cell types; and comparing the images obtained of the multiplicity of first reference cell types to the images obtained of the multiplicity of second altered test-cell types to identify assay response image changes that differ between the second test-cell types, wherein assay response image changes correspond to the biological mechanisms affected by the selected gene in the particular cell type.

28. A method of producing a fingerprint of assay responses caused by a perturbation, comprising
a) culturing a first reference cell under reproducible conditions;
b) processing the first reference cell through a multiplicity of assay experiments in the absence of a perturbation;
c) collecting one or more images of the first reference cell to detect a first cell assay response to the respective assays;
d) culturing a second test-cell under the reproducible conditions of step a), wherein the first reference cell and the second test-cell are the same cell species;
e) processing the second test-cell through the same multiplicity of assay experiments of step b) in the presence of a perturbation;
f) collecting one or more images of the second test-cell to detect a second test-cell assay response to the respective perturbation;
g) comparing the one or more images obtained of the first reference cell to the one or more images obtained of the second test-cell to identify assay response image changes between the first reference cell and the second test-cell, wherein the assay response image changes correspond to a fingerprint of assay responses caused by the perturbation.

29. The method of claim 28, further comprising repeating steps a) through g); with a multiplicity of perturbations.

30. The method of claim 29, further comprising identifying shared patterns of assay response image changes between the multiplicity of perturbations and identifying within the shared patterns, a specific sub-pattern of assay response image changes, wherein the sub-pattern of assay response image changes corresponds to an individual biological mechanism or a subset of all biological mechanisms affected by the subgroup of perturbations.

31. The method of claim 30, wherein the specific sub-pattern of assay response image changes is identified using one or more statistical clustering methods.

32. The method of claim 31, wherein the one or more statistical clustering methods is selected from the group consisting of fuzzy-clustering and multi-domain clustering.

33. The method of claim 28, wherein the perturbation is selected from any one or more of the forces selected from the group consisting of chemical, biological, mechanical, thermal, electromagnetic, gravitational, nuclear, and temporal.

34. The method of claim 33, wherein the perturbation is treatment with a test-compound.

35. The method of claim 34, wherein the test-compound is known to modulate one or more known biological mechanisms.

36. The method of claim 29, wherein the multiplicity of perturbations is treatment of the cells with a multiplicity of test-compounds.

37. The method of claim 36, wherein the multiplicity of test-compounds are each knows to modulate one or more known biological mechanisms.

38. The method of claim 28, wherein the first reference cell is labeled with one or more imaging reagents corresponding to the respective assay, and wherein the second test-cell is labeled with the same one or more imaging reagents of step b).

39. The method of claim 28, wherein steps a) through g) are repeated for a multiplicity of different imaging reagents.

40. The method of claim 28, wherein a series of images are collected over time to assess the temporal behavior of the first and second cells.

41. The method of claim 40, wherein the images are collected after multiple times, during the same assay experiment.

42. The method of claim 41, wherein the cells are fixed prior to collecting the images.

43. The method of claim 40, wherein the images are collected at different times on different assay experiments of the same cell species.

44. The method of claim 28, wherein the images collected are of different assay experiments of same cell type subject to the same perturbation at different quantities.

45. The method of claim 44, wherein the perturbation is a test-compound administered at different concentrations.

46. The method of claim 28, wherein the images are collected from different locations within the first and second cells.

47. The method of claim 28, wherein the images are collected from different locations within the first and second cells.

48. The method of claim 28, wherein the images are collected from different locations within the assay container containing the first and second cells.

49. The method of claim 28, wherein the first and second cells are cell lines.

50. The method of claim 28, wherein the assay response image changes are associated with the respective perturbation and stored in a database.

51. An imaging device suitable for conducting the method of claim 1.

52. An imaging device suitable for conducting the method of claim 28.
